(19) United States
(12) Patent Application Publication
(10) Pub. No.: US 2026/0124415 A1
(43) Pub. Date: May 7, 2026

(54) Method and Apparatus for Monitoring Oxygen Devices

(71) Applicant: Pure O2 Ltd, Urmston (GB)

(72) Inventors: Rupak Kharel, Lancashire (GB); Khaled Rabie, Manchester (GB); Peter Gough, Manchester (GB); Corrado Puntillo, Manchester (GB); Richard Blackhurst, Manchester (GB); Tom Liptrot, Manchester (GB)

(21) Appl. No.: 19/118,198

(22) PCT Filed: Oct. 3, 2023
(86) PCT No.: PCT/GB2023/052554

§ 371 Date: Apr. 3, 2025

(30) Foreign Application Priority Data
Oct. 3, 2022 (GB) 2214526.2

(51) Int. Cl. A61M 16/00; A61M 16/06

(52) U.S. Cl. CPC A61M 16/024; A61M 16/0003; A61M 16/0672; A61M 2016/0027; A61M 2205/13; A61M 2205/18

(57) ABSTRACT

Described herein are methods and devices for monitoring oxygen delivery to a patient using an oxygen supply system. The method includes receiving air flow pressure data, the air flow pressure data indicating pressure measured in a flow channel of a delivery device configured to be connected to an oxygen supply device for delivering oxygen to the patient in a predetermined time period; processing the air flow pressure data to determine one or more oxygen delivery states associated with the air flow pressure data; and monitoring oxygen delivery to the patient based upon the one or more oxygen delivery states.

### FIELD

This description relates to systems, methods, and apparatus for oxygen provision and monitoring. Specifically it relates to monitoring oxygen delivery to a person, and/or monitoring properties of the delivery.

### BACKGROUND

The treatment of many respiratory diseases with oxygen provision is widespread and increasing. In order to monitor the oxygen provision and patient safety, real-time monitoring of the oxygen supply is desirable. However, few technologies are currently available that enable such monitoring, and those technologies that are available are limited in their application. In applications where the provision requires the use of a mask or nasal cannula, oxygen provision at the patient location is often unchecked. Furthermore, in the case where the oxygen mask or the nasal cannula is accidentally disconnected during use, existing technologies do not currently offer a solution to detect this. The systems, apparatus, and methods described herein aim to improve aspects of oxygen delivery, and address at least some of the challenges set out herein.

### SUMMARY

According to a first aspect of the present disclosure there is provided a method of monitoring oxygen delivery to a patient using an oxygen supply system. The method comprises receiving air flow pressure data in a predetermined time period. The air flow pressure data indicates a pressure measured in a flow channel of a delivery device configured to be connected to an oxygen supply device, wherein the oxygen supply device is configured to deliver oxygen to the patient. The air flow pressure data is processed to determine one or more oxygen delivery states associated with the air flow pressure data. Oxygen delivery to the patient is monitored based upon the one or more determined oxygen delivery states.

Optionally, the method further comprises determining, based on the one or more oxygen delivery states, whether the air flow pressure data is indicative of a breathing pattern.

Optionally, the method may further comprise determining, if the air flow pressure is not indicative of a breathing pattern, that the oxygen supply device is disconnected from the patient. In response to the determination that the delivery device is disconnected, a signal may be output comprising a notification that the delivery device is disconnected from the patient.

Optionally, the delivery device may comprise a nasal cannula configured to be attached to the nose of the patient, e.g. through insertion of a prong of the cannula into a part of one or both nostrils of the patient. Alternatively, the delivery device may comprise a face mask configured to be placed over the mouth and/or nose of the patient.

Optionally, the delivery device may comprise first and second flow channels. The oxygen supply device may be connected to the first flow channel. A pressure sensor configured to measure the air flow pressure data may be configured to be connected to the second flow channel. The first and second flow channels may combine inside the delivery device prior to the output of the delivery device, such that fluid can flow from both the first and second channels to the patient. A delivery device comprising first and second flow channels may be referred to as a dual cannula.

Optionally, the delivery device may comprise a first flow channel. The oxygen supply device may be connected to the first flow channel. The pressure sensor configured to measure air flow pressure data may be configured to be connected to the first flow channel, for example via a branch connection (e.g. a T-branch connection). The first flow channel may be a single flow channel (i.e. no other fluid flow channels may be provided between the oxygen supply device and the patient). A delivery device comprising a single flow channel may be referred to as a single cannula.

Optionally, processing the air flow pressure data may comprise denoising the received air flow pressure data.

Optionally, denoising the air flow pressure data may comprise removing a high frequency component related to oxygen supplied by the oxygen supply device.

Optionally, processing the air flow pressure data may further comprise determining at least one parameter from the air flow pressure data, the at least one parameter selected from the group consisting of: slope, amplitude, and frequency. The one or more oxygen delivery states may be determined based on the at least one parameter.

Optionally, the oxygen delivery states may comprise one or more of:
“delivery device connected”, “delivery device disconnected”, “oxygen supply device connected”, “oxygen supply device disconnected”, “normal patient breathing pattern”, and “abnormal patient breathing pattern”.

Optionally, denoising the air flow pressure data may comprise processing the air flow pressure data based upon a threshold value.

Optionally, the method may further comprise receiving oxygen concentration data. The oxygen concentration data may indicate an oxygen concentration in the delivery device. Specifically, the oxygen concentration may be measured in a flow channel connecting the oxygen supply device to the delivery device. The concentration may be measured at a position along the flow channel that is close to the patient (e.g. within 1 m, 2 m, 3 m from the patient). Alternatively or additionally, the method may comprise receiving oxygen flow data.

Optionally, the method may further comprise processing the oxygen concentration data to determine the one or more oxygen delivery states associated with the air flow pressure data.

Optionally, the method may further comprise determining a signal associated with the oxygen supply device, and determining connection to the oxygen supply device based on the signal associated with the oxygen supply device. The signal may be based on the air flow pressure data, oxygen concentration data, or a combination of both.

Optionally, processing the air flow pressure data may comprise processing the air flow pressure data by a machine learning model trained to analyse the air flow pressure data.

Optionally, the machine learning model may be further trained to analyse the oxygen concentration data.

Optionally, monitoring oxygen delivery to the patient may comprise outputting an alert.

Optionally, the alert may comprise at least one of a textual alert, an audio alarm, and a visual alarm.

It will be appreciated that in normal operation the delivery device is connected to the oxygen supply device, however the delivery device may become disconnected from the oxygen supply device in use and while air flow pressure data is obtained. The air flow pressure data may therefore be obtained both when the delivery device is connected to the oxygen supply device, and when the delivery device is disconnected from the oxygen supply device.

According to another aspect of the current disclosure, there is provided a device for monitoring oxygen quality at a patient using an oxygen supply device. The device comprises an input module, comprising at least one input port for connecting to a delivery device for delivering oxygen from an oxygen supply device to a patient. The device further comprises a pressure sensor, connected to the at least one input port, and configured to measure air flow pressure data received through the at least one input port. The device comprises a processor, configured to perform a method as set out above. The device further comprises an output module, configure to provide an output based on the one or more oxygen delivery states determined by the processor.

Optionally, the output module may comprise a wireless transmitter configured to send the signal to a receiver external to the device.

Optionally, the device may further comprise an oxygen concentration sensor connected to the at least one input port, and configured to measure oxygen concentration data at the at least one input port. Optionally, the oxygen concentration sensor may be an oxygen concentration and flow sensor.

### BRIEF DESCRIPTION OF THE FIGURES

Embodiments of the disclosure will now be described, by way of example only, with reference to the accompanying figures, in which:

FIG. 1 depicts a schematic representation of a device for oxygen provision monitoring;

FIG. 2 depicts a schematic representation for oxygen delivery and monitoring;

FIG. 3 depicts a flow chart showing processing to monitor oxygen delivery;

FIG. 4 depicts a schematic representation of a system for oxygen delivery and monitoring using a dual cannula delivery device;

FIG. 5 depicts a flow chart showing processing to determine one or more oxygen delivery states;

FIG. 6 depicts a schematic representation of a system for oxygen delivery and monitoring using a single cannula delivery device; and

FIG. 7 depicts a system for determining an oxygen delivery state using a machine learning model.

### DETAILED DESCRIPTION

Described herein are systems, methods, and apparatus for monitoring oxygen provision to a person. The person may be a patient receiving oxygen supply as part of a therapy. The system may include remote monitoring system implementation, such that the oxygen delivery to the patient may be monitored remotely from the patient. These features may provide advantages by enabling a means to monitor patients in care, at home, or in medical settings remotely, for example by care/medical staff, or the patient themselves. It may also be used for remote monitoring of the equipment in the system, by technicians.

The system may include a real-time alarm to indicate an issue with the oxygen supply to the person. Such a real-time alarm, either remotely or at the patient site, may have an advantage of enabling a prompt response to check for potential issues with the delivery of oxygen. Example issues include, but are not limited to mask/cannular disconnection, oxygen flow problems, oxygen purity drops, and/or patient breathing issues. The alarm may thereby improve patient care and safety, and may potentially save lives.

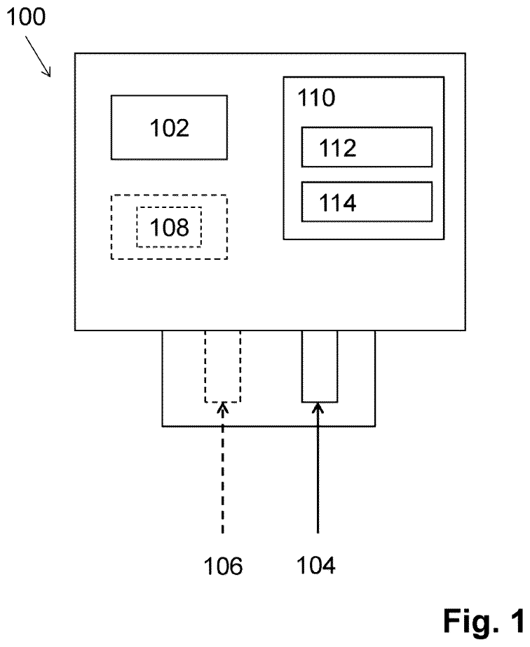

The oxygen provision/delivery, and monitoring obtains data from a device comprising one or more sensors. The device is configured to provide real-time monitoring of oxygen delivery to a patient based on sensed data, as depicted in FIG. 1. The oxygen sensing device 100 may also be referred to as a sensing device, a monitoring device, or an oxygen monitoring device. The sensing device 100 comprises an input module having one or more input ports configured to be connected to one or more external devices as described herein. Device 100 is configured to be connected by a connection 104 to a patient receiving an oxygen treatment The connection 104 may be along an air flow channel (e.g. tube) connected to the patient's breathing output (e.g. connected to mouth and/or nose). The sensing device 100 may comprise a pressure sensor 102 configured to detect pressure inside the patient air flow channel 104. The sensing device 100 may additionally or alternatively comprise an oxygen concentration sensor 108 (or oxygen concentration and flow sensor), for example for determining a quality of the supplied oxygen. In some implementations, the sensing device 100 may comprise a connection 106 to an oxygen supply system, e.g. to an oxygen supply line.

The sensing device may further comprise a controller 110 comprising one or more processors 112 and memory 114 for storing software comprising instructions to operate the device 100 as described herein. The sensing device may also comprise an output module, including wired connectors and/or wireless transmitters/receivers for providing output data determined by the device. The pressure sensor 102 may or may not be a differential pressure sensor. In case of a differential pressure sensor, the reference pressure may be connected to the atmosphere.

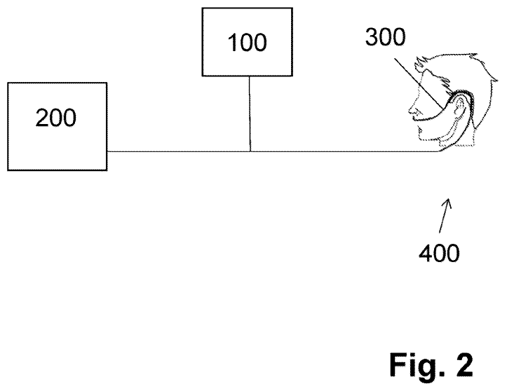

FIG. 2 depicts a schematic representation of a system for oxygen delivery and monitoring. A patient 400 is connected to an oxygen supply device 200. The oxygen supply device 200 (e.g. an oxygen concentrator) is configured to provide a flow of oxygen to the patient 400 via an oxygen delivery device 300. The delivery device 300 may be connected to the patient's nose and/or mouth. The delivery device may be a cannula with prongs configured to be inserted into a part of the patient's nostrils, or another connector from the air flow channel to a patient's nose and/or mouth (e.g. a face mask). Oxygen sensing device 100 is connected to the delivery device in order to monitor oxygen delivery to the patient 400.

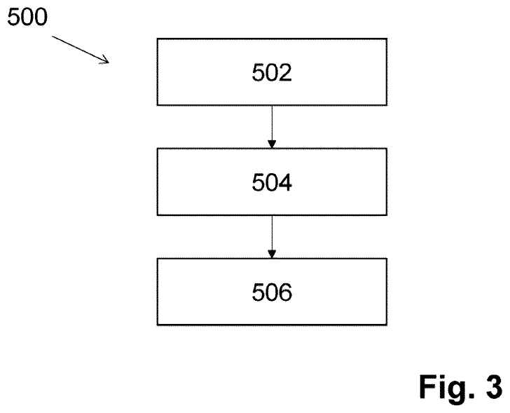

Disclosed herein is a method 500 of monitoring oxygen delivery to a patient as shown in FIG. 3. The oxygen delivery is performed using an oxygen supply system. In step 502, air flow pressure data is received. The air flow pressure data indicates a pressure measured in a flow channel (e.g. pipe) configured to be connected, in use to a delivery device (e.g. a cannula). The delivery device is configured to be connected, in use, to an oxygen supply device (e.g. an oxygen concentrator). The oxygen supply device is configured to, in use, supply an oxygen flow to a patient via a flow channel. The flow channel connected to the pressure sensor may be the same or different to the flow channel connected to the oxygen supply device. The air flow pressure data may be provided for a predetermined time period during which pressure is measured. The air flow pressure data is processed at step 504, to determine one or more oxygen delivery states associated with the air flow pressure data. The oxygen delivery states may, for example, indicate connection or disconnection of the delivery device, and/or a breathing state of the patient. Based on the oxygen delivery states, the oxygen delivery to the patient is monitored 506.

An advantage of the method according to FIG. 3 is that it can provide real time monitoring of the oxygen delivery to a patient. The processing of the air flow pressure data may have a computational cost that is sufficiently low to enable real time determination of a breathing pattern, based on the oxygen delivery states.

The method may comprise providing an output. The output may comprise one or more of an alert, data (e.g. oxygen delivery state(s)), and display (e.g. displaying the determined breath pattern). Options for output provided by the method are described in more detail below. The method may comprise outputting the one or more oxygen delivery states. The one or more oxygen delivery states may be a plurality of oxygen delivery states determined for a time period that together provide breathing pattern data indicating a breathing pattern of the patient over the time period. The one or more oxygen delivery states and/or breathing pattern data may be output substantially in real-time. The output may be provided locally (e.g. to a display). Outputting the data may alternatively and/or additionally comprise transmitting the data wirelessly. Alternatively, or additionally, the data may be output via a wired connection.

Monitoring oxygen delivery to the patient based upon the one or more oxygen delivery states may comprise determining that the air flow pressure data received by the monitoring device 100 is indicative of substantially no breathing pattern (or a breathing pattern with an amplitude below a threshold value). Based upon this air flow pressure data, the processing may determine an oxygen delivery state indicating that the delivery device is disconnected from the patient. This may for example be because a cannula (or mask) has been disconnected from the patient's nose and/or mouth.

Monitoring oxygen delivery to the patient based upon the one or more oxygen delivery states may comprise determining that the air flow pressure data is indicative of substantially no oxygen delivery to the delivery device 300 and/or patient 400, or oxygen delivery to the delivery device 300 and/or patient 400 that is less than a threshold oxygen delivery. The processing may, for example, determine an oxygen state indicating that the oxygen supply device 200 is disconnected from the delivery device 300 and/or patient 400. The method may further comprise outputting a signal. The signal may comprise a notification that the delivery device may be disconnected from the patient.

In some implementations the monitoring device may comprise an oxygen concentration sensor. In such implementations, the method 500 may further comprise receiving oxygen concentration data, for example as measured by the oxygen concentration sensor. The oxygen concentration data may be processed, together with or separately from the air flow pressure data, in order to determine one or more oxygen delivery states. Oxygen delivery states that are based at least in part on oxygen concentration data may include for example indicating that the delivery device is disconnected, indicating that the oxygen supply device is disconnected, and/or indicating that insufficient oxygen is delivered, for example an oxygen concentration below a threshold value. In some implementations, the oxygen concentration sensor may be an oxygen concentration and flow sensor. Oxygen concentration data as described herein, may in those instances be understood to comprise oxygen concentration and oxygen flow data.

The oxygen monitoring device may differentiate between different types of disconnections. A first type is delivery device disconnection, also referred to as cannula disconnection. Delivery device disconnection may occur when the cannula (or other type of delivery device) disconnects from the patient's nostrils, so that the patient cannot receive oxygen, The rest of the oxygen monitoring system may still be functioning correctly, but the patient will not be provided with oxygen. A second type of disconnection is oxygen disconnection, where the cannula is disconnected from the oxygen supply (or the oxygen supply is faulty). In this case, no oxygen is supplied to the cannula, so even when the patient is wearing the device correctly, no oxygen is supplied. Data from the oxygen concentration (and flow) sensor may for example be used to determine an oxygen delivery state that indicates disconnection of a an oxygen supply from the device.

An advantage of the monitoring of oxygen concentration may be that the delivered oxygen concentration may be monitored substantially in real time. Oxygen concentration information may be provided alongside air flow pressure data to determine the oxygen delivery states.

Oxygen concentration may be a parameter describing the amount of oxygen supplied by a concentrator to the patient in an amount of time (e.g. as litres per minute). An oxygen concentration sensor may be able to measure the concentration of oxygen in the air flow, for example in a range from 0% to 100%. The oxygen flow sensor may be able to measure a flow in a range of 0-15 Lpm, or another range relevant to the application for which the sensor is provided. The measured concentration and/or flow may be received, and processed, as part of a method for determining a quality of the oxygen flow. Determining a quality of the oxygen flow may form part of the method of monitoring oxygen supply described herein.

In response to determining one or more oxygen delivery states (associated with air flow pressure data and/or oxygen concentration data), the monitoring device may issue an alert. The alert may comprise an indication of the one or more associated oxygen delivery states. For example, if an oxygen delivery state is determined that indicates disconnection of a cannula from a patient's face, the monitoring device may issue a textual notification that the cannula has disconnected. Alternatively and/or additionally to a textual notification, the alert may comprise an alarm, The alarm may comprise one or both of an audio alarm, and a visual alarm. The notification and/or alarm may be provided at a patient site (e.g. a buzzer and/or LED at the monitoring device 100), and/or remotely. If provided remotely, the monitoring device may transmit the alert to the remote interface, via wireless and/or wired connection.

The one or more oxygen delivery states, and/or the alert may be sent to an interface remote to the patient site. In some embodiments the air flow pressure data and/or oxygen concentration data may also be transmitted. Example interfaces include, but are not limited to an app on a computing device, such as a smartphone, tablet, or other personal computing device, or a web page for display on a computing device. The interface may provide a visual display of the one or more oxygen delivery states (e.g. as a textual notification, a graph, etc). In some implementations the interface may receive an alert from the monitoring device. In some implementations, the interface may be configured to produce an alert in response to receiving the oxygen delivery state(s).

In some implementations, the alert may be determined to be urgent. An urgent alert may be configured to be transmitted to the computing device regardless of whether the breathing pattern is being visually displayed. This may for example be the case when an oxygen delivery state indicates a disconnection of the delivery device, a lack of breathing pattern detection, etc. One or both of the monitoring device and remote interface may be configured with selection rules to determine whether or not an alert is urgent.

In some implementations, in response to an oxygen delivery state being determined, the system may make one or more adjustments to the settings of the oxygen delivery. For example, the oxygen flow and/or oxygen concentration may be reduced, or increased. The adjustment of the setting may be determined (e.g. calculated) by the system, based on the determined oxygen delivery state. In some instances, other information may be used as well, e.g. air flow pressure measurement data, oxygen concentration measurement data, and/or any processed version of that data. In some implementations an adjustment may be selected from a predetermined list of adjustments. The selection may be made based on the determined oxygen delivery state. The predetermined list may have been created using general medical data and/or patient-specific data, and known oxygen delivery treatment dosages.

The air flow pressure data may be processed by a model. The model may comprise one or more algorithms. In some instances, the model may comprise a machine learning model trained to analyse air flow pressure data and/or oxygen concentration data. Example models for processing air flow pressure data are described below. A machine learning model may be used to detect and classify oxygen delivery states. The detection and classification may be performed substantially in real time (e.g. within a few seconds or minutes of the oxygen delivery state occurring).

In an example implementation, the oxygen delivery device may be a nasal cannula. The nasal cannula may, in use, be connected to a patient's nose, e.g. inserted into one or both nostrils. The nasal cannula may comprise a pipe connectable to an oxygen supply device for supplying a flow of oxygen to the patient. The nasal cannula may further be connected to the monitoring device. As the patient breathes, the breathing pattern may cause variations in air flow in the pipe of the cannula. Therefore, air flow inside the cannula may comprise components related to patient breathing and/or oxygen supply flow from the oxygen supply device.

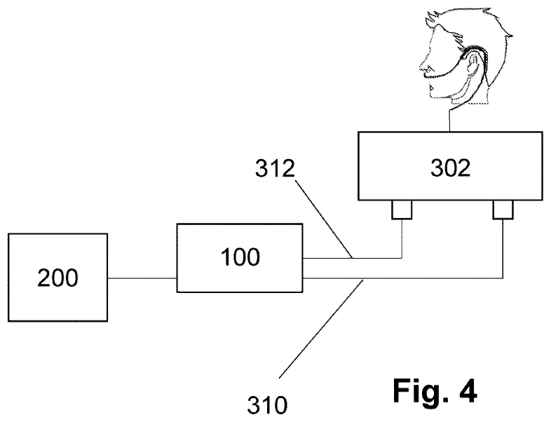

In a first example, the nasal cannula may be a dual cannula 302, as depicted in FIG. 4. A dual cannula may have first and second flow channels through which separate flows may be connected to the cannula. The flows from the first and second channels may be combined (i.e. passively mixed) inside the cannula, before being provided to the patient (e.g. via prongs inserted in the patient's nostrils). This way, flow coming from both the first and second channels may be provided into both nostrils. Air flow provided into the first channel towards the cannula prongs is not present in the second channel and vice versa. However, any breath into the prongs of the cannula will be present in both the first and second channels.

The dual cannula 302 may comprise first 310 and second 312 pipes (providing flow channels through which fluid can flow). The first and second pipes connect into a single channel prior to connection to the patient. That is, air delivered to the patient is delivered by way of a single channel that is connected to both of the first and second pipes. The first pipe 310 may be connected to the oxygen supply device 200 for providing oxygen flow to the patient. The second pipe 312 may be connected to the pressure sensor of the monitoring device 100. The second pipe 312 may be free from oxygen flow coming from the oxygen supply device 200. The breathing pattern from the patient may be present in both the first 310 and second 312 pipes. It will be appreciated that while the first pipe 310 is shown as not connected to the monitoring device 100 in FIG. 4, in some embodiments the second pipe may also be connected to the monitoring device 100, for example connected to oxygen concentration sensor 108 of the monitoring device 100.

In an example implementation, the first pipe of a dual cannula may be used to deliver oxygen to a patient, while the second pipe may be used to detect the air flow pressure from the patient's breathing. The monitoring device 100 may have a first channel which has a first end connected to an oxygen supply device, and a second end connected to the first pipe of the dual cannula. This first channel may be sued for oxygen delivery, and optionally for measurements of oxygen concentration and/or oxygen flow. The monitoring device may have a second channel used to measure pressure from the second pipe 312 of the dual cannula 302.

In case of a single cannula, the cannula may have a single pipe. The single pipe may be used for oxygen supply and air flow pressure measurement. The monitoring device may have a single channel which has a first end connected to an oxygen supply device, and a second end connected to the single pipe of the single cannula. An internal T-connection may be present along the single channel of the monitoring device 100. Air flow pressure may be measured from this T-connection.

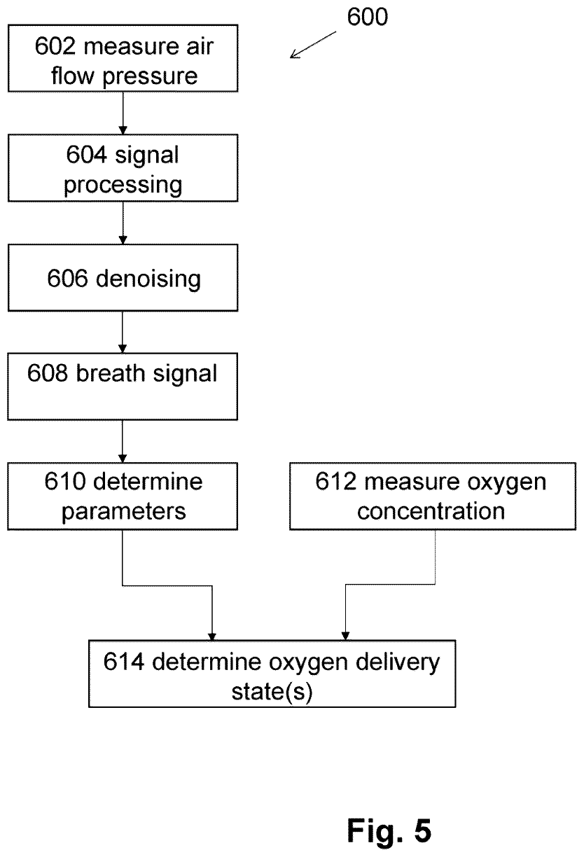

An example process 600 of determining oxygen delivery states using a cannula is depicted in FIG. 5. The variations in air flow in the second pipe 312 may be measured 602 by a pressure sensor of monitoring device 100. The pressure sensor may measure sample air flow pressure measurements over a predetermined period of time. The sampled air flow pressure measurements may be processed 604 to form an air flow pressure signal. The signal may comprise a breathing pattern of the patient, and/or one or more noise components.

In some implementations, as part of processing the air flow pressure data, the processor may perform a denoising step 606. As the oxygen supply flow runs through a separate pipe to the cannula and no substantial oxygen flow pattern is present in the second pipe, the denoising may be relatively straightforward, compared to configurations where the breathing pattern is detected in the same pipe that carries an oxygen supply. In some instances using a dual cannula, the denoising step may be omitted. Isolating the breathing pattern from the air flow pressure signal through denoising may result in obtaining a breathing pattern signal 608, which may be referred to as a breath signal.

For denoising, when a dual cannula is used the air flow pressure signal is typically already centered around zero, and no baseline drift is present, and therefore does not present any external noise contribution. The denoising may for example comprise a low-pass filter, which may reduce internal noise caused by electrical connections. This may improve the signal provided by the pressure sensor. When a single cannula is used, denoising may involve centering around zero, as well as improving the quality of the signal (e.g. using a low-pass filter).

Once the breathing pattern signal 608 has been obtained from the air flow pressure data, the processing may involve signal analysis 610 to determine one or more parameters of the breathing pattern signal. the parameters may include frequency, amplitude, and slope of the signal. The analysis may determine one, two, or all three of these parameters. Other parameters related to the signal may be determined in addition to or as an alternative to the parameters listed above. The parameters may be obtained from the signal through mathematical methods (e.g. derivative computation, local maximum/minimum determination, etc.) Optionally, the process may involve obtaining oxygen concentration measurements 612. An oxygen concentration sensor may measure sample oxygen concentration measurements over a predetermined period of time. The sample rate and/or predetermined period of time may be the same as for the air flow pressure measurements of step 602. The oxygen concentration may represent an oxygen concentration in an air flow channel provided with oxygen by the oxygen supply device.

Based on the determined parameters, at step 614 one or more oxygen delivery states may be determined. For example, the signal analysis may detect a breathing pattern within the expected breathing range, indicating nasal cannula connection. If no breathing pattern is detected in the signal analysis, this may be an indication of cannula disconnection.

In order to determine whether or not a signal constitutes a breath pattern, one or more threshold values may be used (e.g. in the denoising step and/or in the determination whether the resulting breath pattern components is indicative of patient breathing or not). A threshold may be set to differentiate between breath signal from noise in the air flow pressure data. The threshold may be set based on previous knowledge of breathing patterns. The threshold may be set based on the received air flow pressure data. The threshold may alternatively and/or additionally be adjusted based on the received air flow pressure data, over time. The method may comprise an option to manually adjust the threshold, by a user, for example to adjust the sensitivity of the system.

As described above, in some instances, oxygen concentration data may be measured 612 by an oxygen concentration sensor. The oxygen concentration data may be processed together with the air flow pressure data. the oxygen concentration data may be used in the determination 614 of one or more oxygen delivery states, by itself, and/or in combination with the air flow pressure data. For example, in a case where the air flow pressure data indicates a breathing pattern is present, while the oxygen concentration data shows a low concentration of oxygen, this may be an indication of oxygen supply disconnection. In case the oxygen concentration is at expected levels, while no breathing pattern data is detected, this may be an indication of disconnection of the nasal cannula from the patient. In order to measure the oxygen concentration in the first pipe 310, the first pipe 310 may pass through the monitoring device 100. An oxygen concentration sensor may be present in the monitoring device, and may for example be connected to the first pipe 310 by means of a T-connector.

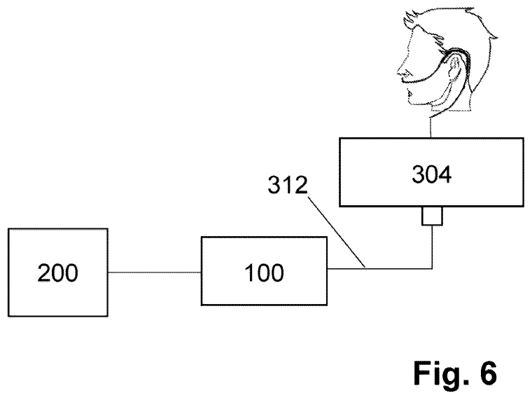

In a second example, the nasal cannula may be a single cannula 304, as depicted in FIG. 6. Unless stated otherwise, any of the features described for the dual cannula may also apply for the single cannula setup. The single cannula may have a single (first) pipe 310, configured to be connected, in use, to an oxygen supply device for providing oxygen flow to the patient. A monitoring device 100 may be connected to the first pipe 310, for example via a T-branch connection. The process of determining one or more oxygen delivery states may be generally the same as described in relation to FIG. 5 and a dual cannula above. For a single cannula, air flow in the first pipe may comprise both an oxygen flow component coming from the oxygen supply device 200, as well as a breathing pattern component breathed into the pipe by the patient.

Due to the presence of both the breathing pattern and oxygen flow components flowing in the channel, a single cannula requires more involved denoising 606, to isolate the breath pattern from the oxygen flow and other noise components. This may present challenges, as the breathing frequency and oxygen supply frequency may have similarities. In some instances, the oxygen supply device may be configured to provide oxygen at a frequency that has a same or similar order of magnitude as the order of magnitude of a standard breathing pattern. For the configurations described herein, when the oxygen supply flow to the patient is higher, the oxygen flow component is stronger inside the pressure signal. As a result, a stronger oxygen flow component may be more challenging to remove from the signal in order to isolate the breath pattern, and the use of more powerful denoising tools (e.g. a machine learning model as discussed below) may be particularly advantageous.

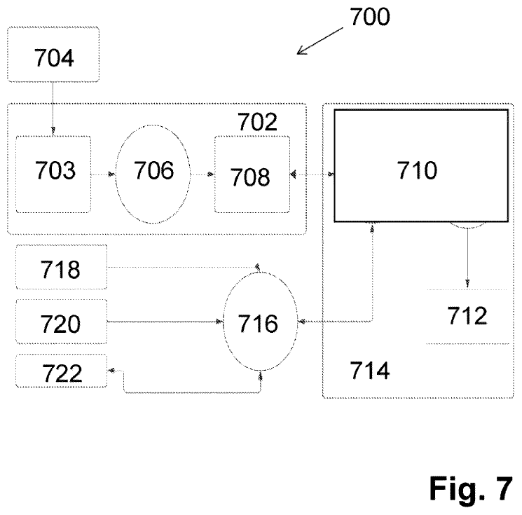

In some implementations, a machine learning model may be used to perform at least a portion of the denoising step 606. The machine learning model may, for example, be trained to obtain data indicative of a breathing pattern from air flow pressure detected by a pressure sensor in the configuration of FIG. 6. The machine learning model may additionally be trained to obtain data indicative of a breathing pattern in a signal comprising an oxygen supply. The machine learning model may initially be trained on generic patient data. The machine leaning model may in some cases be further trained (i.e. finetuned) based on data of the specific patient being monitored. This may involve, for example, continuously or periodically updating the machine learning model based on live and/or recent patient data of the patient being monitored, for example based on data stored in a database as described below. A machine learning model may therefore be updated through training on new/updated patient data, whilst in use performing denoising of patient data. Some or all of the steps 604, 606, 608, and 610 may be performed by a machine learning model, as described for example in relation to FIG. 7 below.

Once the data has been denoised to isolate the breath signal 608, signal analysis may be performed on the signal 610 to determine parameters of slope, amplitude, and/or frequency as described above. Optionally, oxygen concentration data may also be obtained 612. On or both of the oxygen concentration data and the breath signal parameters may be used to determine 614 one or more oxygen delivery states associated with the patient data. The oxygen delivery states may be output and/or otherwise used as described herein.

Many oxygen delivery systems use a single cannula configuration. A further advantage of a single cannula setup may be that it is able to determine oxygen delivery states associated with the oxygen supply device based on only air flow pressure data without requiring oxygen concentration data (e.g. oxygen supply device disconnection). In particular, it will be appreciated that the denoising performed to isolate breath signal at step 608 for a single cannula configuration effectively determines (and removes) a signal component that is attributable to the oxygen delivery device, which can be independently processed to determine one or more states associated with the oxygen delivery device. For example, based on the air flow pressure data, a single cannula configuration may determine that a breath signal is absent while an oxygen flow is present. This may indicate disconnection of the cannula from the patient's nostrils. In another example the monitoring device may determine from the air flow pressure data that a breath pattern is present, while the oxygen flow from the supply device is absent. This may indicate disconnection of the oxygen supply device. In both instances, a separate alarm may be raised, identifying the specific problem.

As stated above, the obtained air flow pressure data may comprise discrete measurement values obtained over a period of time. The measurements may be seen as samples of the patient's breath signal inside the channel. The signal may be sampled using standard signal processing methods. The minimum sampling frequency (the number of measurement points needed per second) is set according to the Nyquist theorem. The minimum sampling frequency should be at least at the Nyquist frequency, that is to say, at least half of the lowest frequency to be detected in the sampling. However, more samples per second are desirable, in order to have more detailed information about the breath signal. A higher sampling rate may offer an advantage of making noise components in the signal more visible in the signal and therefore easier to detect. As a result, signal analysis may allow for a more detailed and accurate reconstruction of the breath signal when the sampling rate is higher. However, the computational cost and measurement requirements become higher as the sampling rate increases. Therefore, a balance may be found between high and low sampling rate to find a balance between breath signal reconstruction detail and computational cost. A sampling frequency may for example be in a range of 3.0 Hz 3.5 Hz. A sampling frequency may preferably be at least 10 times higher than the highest frequency of the signal of interest (i.e. the breath signal). A sampling frequency may preferably be at least 20 times higher than the highest frequency of the signal of interest (i.e. the breath signal).

Denoising may involve the removal of components of the air flow pressure data that are not caused by the patient's breath. Different types of noise may be present in the air flow pressure data. Some components may be low frequency components, for example a baseline drift of the measurement system, environmental effects, etc. Some components may be high frequency components, for example the oxygen supplied by the oxygen supply device The denoising may involve passing the air flow pressure signal through a band pass filter. The bandpass filter may be set to pass a range capturing breathing frequencies. The range of the bandpass filter may for example be set to be or comprise 0.2-0.4 Hz. 0.2 Hz may be an average human breathing frequency at rest. This range may be personalised for a specific patient's breathing frequency.

An example implementation of filters may include for example a series of filtering steps. The steps may for example include: a Butterworth 3rd order low pass filter centred on 0.3 Hz, a notch filter centred on 0.2 Hz, a high pass filter obtained by subtracting a moving average from the breath signal in order to isolate a signal baseline (the moving average may be of the unfiltered air flow pressure signal, and/or a partially filtered version of the air flow pressure signal), and a moving average filter for removing any remaining spikes and smoothing the resulting signal from a partially filtered signal in order to smooth the signal after passing it through the low-pass filter.

The processing step may be performed on a processor provided in the monitoring device. Alternatively or additionally, the air flow pressure data may be transmitted (either wirelessly or wired) to one or more remote processors for remote processing. This may for example be an online application where data is stored and/or processed on the cloud.

When an oxygen concentration analysis is included, the dual cannula may be used to determine disconnection of the oxygen supply device. This may for example be performed by detecting a breathing pattern for the patient, based on the air flow pressure data, while substantially simultaneously detecting a low oxygen concentration using the oxygen concentration monitor.

As described above, in an example implementation an oxygen delivery state may be determined by denoising an air flow pressure data signal, followed by determining a frequency, amplitude, and slope (parameters) of the denoised signal. Based on various predetermined thresholds set for the parameters, an oxygen delivery state is determined. In an alternative implementation, a machine learning model may be used to classify air flow pressure data into one or more oxygen delivery states. The machine learning model may take raw (digitised) air flow pressure data as input (i.e. before a denoising step is performed).

An advantage of using machine learning for determining an oxygen delivery state based on air flow pressure data may be that it can avoid false positives for disconnection compared to a signal denoising and parameter analysis step.

FIG. 7 depicts a system 700 for determining oxygen delivery states using machine learning. A pressure sensor 703 of oxygen monitoring device 702 as described herein may collect air flow pressure data from a patient 704 as described above. This air flow pressure data may be collected using a single cannula. The air flow pressure data may be analogue data. An analog-to-digital converter 706 may convert the analogue data to a digital signal (digitised, sampled). This digitised signal may comprise several samples per second, e.g. 2 Hz-10 Hz, or 3 Hz-20 Hz, such as 4 Hz. Periodically, a processor 708 of the oxygen delivery device 702 may provide the digital signal as an input to the machine learning model 710. This may for example be every 5 second, every 8 seconds, every 10 seconds, or any time in a range of 4 s-20 s. The digital signal comprising samples across a time period may comprise a number of digital samples, and may be referred to as an air flow pressure data packet.

The air flow pressure data packet may be classified by the machine leaning model as an oxygen delivery state. Possible oxygen delivery states include nasal breathing, mouth breathing, disconnected cannula, disconnected oxygen supply, connected oxygen supply, etc. The determined oxygen delivery state may be provided to the processor 708 of the oxygen delivery device 702. The oxygen delivery state and associated raw data may be stored in a database 712. In response to determination of the oxygen delivery state, the oxygen monitoring device may take one or more actions as described herein, such as sending the oxygen delivery state to a device 716. The device 716 may for example be a phone, tablet, computer monitored by a carer, or located in the vicinity of the patient and oxygen monitoring device 702. In response to receiving the oxygen delivery state, the device 716 may sound an audio 718 and/or visual 720 alarm, or generate/display an alert 722.

Other data may be provided to the machine learning model alongside air flow pressure data. This other data may for example include time, date, temperature, oxygen concentration data, etc. This data may be used by the model when determining an oxygen delivery state and/or may be stored alongside the oxygen delivery state and raw data in the database 712.

The machine learning model may be located on a processor outside of the oxygen delivery device, in remote location 714. The processor and memory hosting the machine learning model may for example be a cloud-based processor and memory. This may be due to the size of the model. The database 712 may also be stored in a remote memory 714, such as a cloud-based memory. The processor of the oxygen delivery device may be connected to the remote processor via wired and/or wireless connection(s), using known protocols for communication (e.g. the MQTT protocol).

The machine learning model may be a supervised model, that is to say, the model may be trained on air flow pressure data packets which have been labelled with the correct oxygen delivery status. Training data may have been obtained by collecting air flow pressure data using an oxygen delivery device from a range of different subjects (e.g. different persons). Various oxygen delivery states may have been created for the data (e.g. types of disconnections, nasal/mouth breathing) and the associated air flow pressure data may have been labelled accordingly. The data may then be sampled as described above. The samples data and associated label(s) may be used to train the model. Some of the data may be used for validation.

The air flow pressure data may be pre-processed, for example in each air flow pressure data packet, the median value may be subtracted to centre the signal around zero. In order to reduce the number of false alerts/alarms, an alert/alarm may only be triggered if more than one sequential packet is classified as having the oxygen delivery status associated with the alert.

The machine learning model may be an ensemble time series classification model. The model may include classifiers of one or more domains, such as phase-independent shapelets, bag-of-words-based dictionaries, phase-dependent intervals, etc. The model may be, for example, a Hierarchical Vote Collective of Transformation-based Ensembles described in HIVE-COTE 2.0: a new meta ensemble for time series classification, Middlehurst et. al. which is herein incorporated by reference, and may, for example, be trained using sktime (sktime. net).

The air flow pressure data may be processed by a trained model. The model may be a machine learning model. The model may receive as input air flow pressure data and generate denoised air flow pressure data indicating air flow pressure components attributable to a breathing pattern without air flow pressure attributable to the oxygen supply device 200. The model may for example have been trained on generic air flow pressure data, initially. Once the model is in use, it may receive as input air flow pressure data for a specific patient. The model may be continuously trained/updated based on the patient-specific data to learn patient-specific breath pattern properties. This way, the model may improve patient-specific air flow pressure data analysis, for example for improved breath pattern analysis. In some instances a machine learning model may be used to determine threshold values for determining whether a component of a signal is breath or noise.

Additionally or alternatively, the model may receive the air flow pressure data as input and may process the air flow pressure data in order to produce and provide an output comprising an estimate of one or more oxygen delivery states. That is, in some embodiments the model may replace steps 606 to 614 of FIG. 5. These states may for example include, but are not limited to, breathing normally, cannula connection/disconnection, and oxygen supply device connection/disconnection. Further states may be included, such as for example mouth or nose breathing, coughing, wheezing, and/or shortness of breath. In other instances, the model may be configured to provide as output a denoised breath signal, and further steps may be performed separately from the machine learning model.

Air flow pressure data may be provided as input to the model. Air flow pressure data points may be provided over a period of time to create an air flow pressure signal as a function of time. In a first implementation, air flow pressure data is provided as input one data point at a time, when it becomes available. In a second implementation, input data is provided as a plurality of air flow pressure data points. A plurality of data points may be provided covering a period of time, as a sliding window. In a sliding window, the model may use a sliding window of previously used (old) data points in combination with newly received (not yet used) data points, for determining the output.

The model may provide a breath pattern signal as output. In some implementations, the output may be provided as a single data point at a time, for forming an output breath pattern. Each output data point may for example correspond to an input air flow pressure data point. That is, the output data point may be the determined breath pattern component of a received input air flow pressure data point. In other example implementations, a plurality of output breath pattern points may be provided periodically, as a sliding window for forming a breath signal. The number of output breath pattern data points output per second may be the same as the number of air flow pressure input data points provided to the model.

In some instances, the predetermined time period over which air flow pressure data is provided may be periodically or continuously adjusted, for example to add new air flow pressure data. The new air flow pressure data may be real-time data.

Air flow pressure data may be continuously received and processed and oxygen delivery states may be continuously output. The oxygen delivery states may comprise data indicating a time that a state has changed, and optionally data indicating the state to which the change occurs. Where only two oxygen delivery states are used, only data indicating a time that the state changed may be provided, with prior state data and an indication that the state has changed providing information that allows determination of the new state in such an embodiment. Where oxygen delivery states are provided as an output of a machine learning model, the oxygen delivery states may be provided either as a classification of a current oxygen delivery state, or may alternatively be provided as a likelihood associated with each of the oxygen delivery states.

Oxygen delivery to the patient is monitored based upon the one or more oxygen delivery states. For example, the oxygen delivery states may be monitored to identify one or more states indicating oxygen is not being effectively delivered to the patient. The monitoring may be carried out in a variety of ways. For example, in some embodiments determination that the state has changed from a state indicating that oxygen is being effectively delivered to a state indicating that oxygen is not being effectively delivered to the patient may cause an alert to be output. In other embodiments, an alert may be output if a state indicating that oxygen is not being effectively delivered is determined for a predetermined amount of time.

Additionally or alternatively to providing alerts, the oxygen delivery states may be monitored to determine alternative real-time oxygen provision decisions such as automatically optimising oxygen delivery. For example, where the oxygen delivery states may indicate that oxygen is not being delivered effectively and adjustments of equipment, such as increasing oxygen delivery rate, waking the patient or the like may be made. The oxygen delivery states may additionally or alternatively be used for longer term oxygen provision decisions. For example, the oxygen delivery states may be used to determine to change a delivery device, may be used to change an oxygen provision plan or the like. In addition, oxygen delivery states may be used as biomarker data to monitor a response of the patient to a treatment in clinical trials and/or as part of routine care.

The model may be trained based on training data comprising air flow pressure data in combination with ground truth denoised air flow pressure data. In an example implementation, the ground truth denoised air flow pressure data may, for example, be air flow pressure data obtained using a dual cannula configuration for a patient, which may be aligned with air flow pressure data obtained using a single cannula configuration based on separately acquired breathing pattern data. Alternatively, breathing pattern data may be acquired and used to manually extract breathing pattern data from single cannula air flow data. The training data may be obtained from one or more people other than the patient being monitored. The model may be trained using any suitable model training process that will be understood by a person skilled in the art, for example backpropagation.

Additionally and/or alternatively, the model may be trained and/or updated based on air flow pressure data received as part of the method of monitoring oxygen supply to the particular patient and one or more known oxygen delivery states for the patient. For example, during an initialisation phase a trained model may be provided that has been trained based on one or more patients other than the patient to be monitored. The trained model may be calibrated for the patient by processing air flow pressure data obtained from the patient using the oxygen supply system that is to be used to deliver oxygen to the particular patient, together with known oxygen delivery states. Such calibration data may, for example, be obtained by obtaining air flow pressure data while a delivery device is not delivering oxygen to the patient and by obtaining air flow pressure data while the delivery device is delivering oxygen to the patient. The model may be trained with the calibration data in a corresponding manner to the initial training of the model using data obtained from patients other than the particular patient to optimise the model parameters for the particular patient.

The method may comprise storing the received air flow pressure data at one or more of a local memory and/or at a remote memory (e.g. the cloud). The stored data may be plotted. The stored air flow pressure data may be used to track patient history. The stored data may be used to update the model.

In an example implementation, air flow pressure data may be continuously monitored. The monitoring may take place in the cloud (e.g. cloud based REST API). Received air flow pressure data may be continuously uploaded to a cloud server where it may be cleared, processed, stored, and/or analysed (e.g. processed by a model). Changes in breathing pattern may be monitored, for example using a changepoint detection algorithm. Changepoints may for example be detected using the Pruned Exact Linear Time (PELT) method. Changes of state may be classified, for example using a machine learning algorithm trained on a large sample of real world patient data as described above. As described above, the machine learning model may be fine-tuned to a specific oxygen supply setting, for example to a specific combination of patient and oxygen delivery system. The output of the model may comprise a continuous, real time, best estimate of a state of the system. The system may further provide a probability estimate of each state, which may include a timing for when they started and ended.

In a specific implementation, an oxygen monitoring device may be used in a hospital setting. A simple device may be provided, that is suitable for use with equipment commonly available and in use in hospital settings. This may for example be a device and method using a single cannula oxygen delivery device. As described herein, a single cannula setup may be able to determine both cannula connection/disconnection as well as oxygen supply device connection/disconnection based on the air flow pressure data, and without requiring oxygen concentration data.

In another specific implementation, the oxygen monitoring device may be provided in an at-home care setting (or any real-life care setting away from a health care facility). In such a case, a dual cannula setup may be provided as a solution requiring a simpler denoising process. A single cannula setup would also still work.

A further advantage of the device described herein is that it may be provided close to the patient's side (e.g. close to where the cannula connects to the patient's nostrils). In a real-life care setting (e.g. at home) the oxygen delivery device may be provided in a set location, while a patient is able to move to one or more different locations removed from the oxygen supply device (e.g. throughout a house). This may require longer cable connections to be present between the different locations. By having a device near to the patient that is able to detect oxygen supply disconnection (single cannula based on air flow pressure data, dual cannula based on air flow pressure data and oxygen concentration data). this means that a failure is detected at the patient site as opposed to at the. This means failures along the air flow channel may also be detected right up until the point where it reaches the patient., which would not be the case if oxygen supply was monitored at the supply device itself.

Although described here in relation to oxygen provision, the methods, systems, and apparatus described herein may also be used for the provision of other gases and/or gaseous mixtures as well.

1. A method of monitoring oxygen delivery to a patient using an oxygen supply system, the method comprising:
receiving air flow pressure data, the air flow pressure data indicating pressure measured in a flow channel of a delivery device configured to be connected to an oxygen supply device for delivering oxygen to the patient in a predetermined time period;
processing the air flow pressure data to determine one or more oxygen delivery states associated with the air flow pressure data; and
monitoring oxygen delivery to the patient based upon the one or more oxygen delivery states.

2. The method according to claim 1, further comprising:
determining, based on the one or more oxygen delivery states, whether the air flow pressure data is indicative of a breathing pattern.

3. The method according to claim 2, further comprising
determining, if the air flow pressure is not indicative of a breathing pattern, that the oxygen supply device is disconnected from the patient; and
outputting a signal comprising a notification that the delivery device is disconnected from the patient.

4. The method according to claim 1, wherein the delivery device is a nasal cannula configured to be attached to the nose of the patient.

5. The method according to claim 1, wherein the delivery device comprises first and second flow channels; and
wherein the oxygen supply device is configured to be connected to the first flow channel of the dual cannula, and wherein a pressure sensor configured to measure the air flow pressure data is connected to the second flow channel of the dual cannula.

6. The method according to claim 1, wherein the delivery device comprises a first flow channel; and
wherein the oxygen supply device is configured to be connected to the first flow channel, and wherein a pressure sensor configured to measure air flow pressure data is connected to the first flow channel.

7. The method according to claim 1, processing the air flow pressure data comprises:
providing the air flow pressure data as input to a machine leaning model; and
receiving the one or more oxygen delivery states as output from the machine learning model.

8. The method according to claim 7, wherein the machine learning model is a classifier.

9. The method according to claim 7, wherein processing the air flow pressure data further comprising:
sampling the air flow pressure data at a frequency in range of 2 Hz-10 Hz, or 4 Hz.

10. The method according to claim 9, wherein a plurality of samples are grouped together in an air flow pressure data packet, and wherein the air flow pressure data packet is provided as the input to the machine learning model.

11. (canceled)

12. (canceled)

13. (canceled)

14. The method according to claim 1, wherein the oxygen delivery states comprise one or more of delivery device connected, delivery device disconnected, oxygen supply device connected, oxygen supply device disconnected, normal patient breathing pattern, and abnormal patient breathing pattern.

15. (canceled)

16. The method according to claim 1, further comprising receiving oxygen concentration data, the oxygen concentration data indicating an oxygen concentration in the delivery device.

17. The method according to claim 16, further comprising processing the oxygen concentration data to determine the one or more oxygen delivery states associated with the air flow pressure data.

18. The method according to claim 1, further comprising determining a signal associated with the oxygen supply device, and determining connection to the oxygen supply device based on the signal associated with the oxygen supply device.

19. The method according to claim 1, wherein processing the air flow pressure data comprises processing the air flow pressure data by a machine learning model trained to analyse the air flow pressure data.

20. The method according to claim 19, further comprising receiving oxygen concentration data, the oxygen concentration data indicating an oxygen concentration in the delivery device, wherein the machine learning model is further trained to analyse the oxygen concentration data.

21. The method according to claim 1, wherein monitoring oxygen delivery to the patient comprises outputting an alert.

22. (canceled)

23. A device for monitoring oxygen quality at a patient using an oxygen supply device, comprising:
an input module, comprising at least one input port for connecting to a delivery device for delivering oxygen from an oxygen supply device to a patient;
a pressure sensor, connected to the at least one input port, and configured to measure air flow pressure data through the at least one input port;
a processor, configured to perform a method according to claim 1; and
an output module, configure to provide an output based on the one or more oxygen delivery states determined by the processor.

24. The device according to claim 20, wherein the output module comprises a wireless transmitter configured to send the signal to a receiver external to the device.

25. The device according to claim 23, further comprising an oxygen concentration sensor connected to the at least one input port, and configured to measure oxygen concentration data at the at least one input port.
